(19) United States
(12) Patent Application Publication
(10) Pub. No.: US 2019/0070421 A1
(43) Pub. Date: Mar. 7, 2019

(54) Supercapacitor-Powered Charger and Implantable Medical Device

(71) Applicant: Boston Scientific Neuromodulation Corporation, Valencia, CA (US)

(72) Inventor: Joey Chen, Valencia, CA (US)

(21) Appl. No.: 16/059,508

(22) Filed: Aug. 9, 2018

Related U.S. Application Data
(60) Provisional application No. 62/554,913, filed on Sep. 6, 2017.

(51) Int. Cl. A61N 1/372; A61N 1/05; A61N 1/378

(52) U.S. Cl. CPC A61N 1/37211; A61N 1/375; A61N 1/378; A61N 1/0504

(57) ABSTRACT

A system for providing therapy to a patient using an implantable medical device (IMD) and an external charger for charging the IMD is disclosed. The external charger and/or the IMD are powered using supercapacitors, which have much higher power densities and discharge rates than comparably sized batteries. Thus, the process of charging the IMD with the external charger requires only a short amount of time, for example one to two minutes. The IMD may include a hybrid power system including both a supercapacitor and a rechargeable battery. With such a hybrid power system, the IMD's supercapacitor may be charged very quickly. Subsequently, power stored within the supercapacitor can be used to recharge the rechargeable battery at a slower charging rate.

### CROSS-REFERENCE TO RELATED APPLICATIONS

This is a non-provisional of U.S. Provisional Patent Application Ser. No. 62/554,913, filed Sep. 6, 2017, which is incorporated by reference in its entirety, and to which priority is claimed.

### FIELD OF THE INVENTION

The present invention relates to wireless external chargers and implantable medical device systems.

### INTRODUCTION

Implantable stimulation devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability in any implantable medical device system, including a Deep Brain Stimulation (DBS) system.

As shown in FIGS. 1A-1C, a SCS system typically includes an Implantable Pulse Generator (IPG) 10 (Implantable Medical Device (IMD) 10 more generally), which includes a biocompatible device case 12 formed of a conductive material such as titanium for example. The case 12 typically holds the circuitry and battery 14 (FIG. 1C) necessary for the IMD 10 to function, although IMDs can also be powered via external RF energy and without a battery. The IMD 10 is coupled to electrodes 16 via one or more electrode leads 18, such that the electrodes 16 form an electrode array 20. The electrodes 16 are carried on a flexible body 22, which also houses the individual signal wires 24 coupled to each electrode. In the illustrated embodiment, there are eight electrodes (Ex) on each lead 18, although the number of leads and electrodes is application specific and therefore can vary. The leads 18 couple to the IMD 10 using lead connectors 26, which are fixed in a non-conductive header material 28, which can comprise an epoxy for example.

As shown in the cross-section of FIG. 1C, the IMD 10 typically includes a printed circuit board (PCB) 30, along with various electronic components 32 mounted to the PCB 30, some of which are discussed subsequently. Two coils (more generally, antennas) are show in the IMD 10: a telemetry coil 34 used to transmit/receive data to/from an external controller (not shown); and a charging coil 36 for charging or recharging the IMD's battery 14 using an external charger, which is discussed in detail later.

FIG. 2 shows the IMD 10 in communication with an external charger 50 used to wirelessly convey power to the IMD 10, which power can be used to recharge the IMD's battery 14. The transfer of power from the external charger 50 is enabled by a primary charging coil 52. The external charger 50, like the IMD 10, also contains a PCB 54 on which electronic components 56 are placed. Again, some of these electronic components 56 are discussed subsequently. A user interface 58, including touchable buttons and perhaps a display and a speaker, allows a patient or clinician to operate the external charger 50. A battery 60 provides power for the external charger 50, which battery 60 may itself may be rechargeable. The external charger 50 can also receive AC power from a wall plug or from a port, such as a USB port. A hand-holdable housing 62 sized to fit a user's hand contains all of the components.

Power transmission from the external charger 50 to the IMD 10 occurs wirelessly and transcutaneously through a patient's tissue 25, via inductive coupling. FIG. 3 shows details of the circuitry used to implement such functionality. Primary charging coil 52 in the external charger 50 is energized via charging circuit 64 with an AC current, Icharge, to create an AC magnetic charging field 66. This magnetic field 66 induces a current in the secondary charging coil 36 within the IMD 10, providing a voltage across coil 36 that is rectified (38) to DC levels and used to recharge the battery 14, perhaps via a battery charging and protection circuitry 40 as shown. The frequency of the magnetic field 66 can be perhaps 80 kHz or so. When charging the battery 14 in this manner, it is typical that the housing 62 of the external charger 50 touches the patient's tissue 25, perhaps with a charger holding device or the patient's clothing intervening, although this is not strictly necessary.

The IMD 10 can also communicate data back to the external charger 50 during charging using reflected impedance modulation, which is sometimes known in the art as Load Shift Keying (LSK). This involves modulating the impedance of the charging coil 36 with data bits (“LSK data”) provided by the IMD 10's control circuitry 42 to be serially transmitted from the IMD 10 to the external charger 50. For example, and depending on the logic state of a bit to be transmitted, the ends of the coil 36 can be selectively shorted to ground via transistors 44, or a transistor 46 in series with the coil 36 can be selectively open circuited, to modulate the coil 36's impedance. Such data can be received at the external charger 50, for example at a telemetry module 53, and subsequently transmitted to the microcontroller 72. LSK communications are described further for example in U.S. Patent Application Publication 2013/0096652.

External charger 50 can also include one or more temperature sensors, i.e., thermistors 71, which can be used to report the temperature (expressed as voltage Vtherm) of external charger 50 to its control circuitry 72, which can in turn control production of the magnetic field 66 such that the temperature remains within safe limits. See, e.g., U.S. Pat. No. 8,321,029, describing temperature control in an external charging device.

A drawback to the transcutaneous inductive charging method described above is that it can take significant time to charge the IMD's battery. Active implantable devices such as spinal cord stimulators are required to be repeatedly charged, typically for several hours a week to maintain charge for delivering therapy. There is thus a need for devices and methods that allow a user to spend less time charging the battery of their IMD.

### BRIEF DESCRIPTION OF THE DRAWINGS

FIGS. 1A-1C show different views of an implantable pulse generator, a type of implantable medical device (IMD), in accordance with the prior art.

FIG. 2 shows an external charger being used to charge a battery in an IMD, while

FIG. 3 shows circuitry in both, in accordance with the prior art.

FIG. 4 illustrates a system for charging an IMD using a supercapacitor-powered external charger and a hybrid power system for an IMD.

FIGS. 5A and 5B illustrate a supercapacitor-powered external charger.

FIG. 6 illustrates a functional schematic for a supercapacitor-powered external charger.

FIG. 7 illustrates power circuitry for a supercapacitor-powered external charger.

FIG. 8 shows an IMD with a hybrid power system.

FIG. 9 shows a functional schematic of an IMD with a hybrid power system.

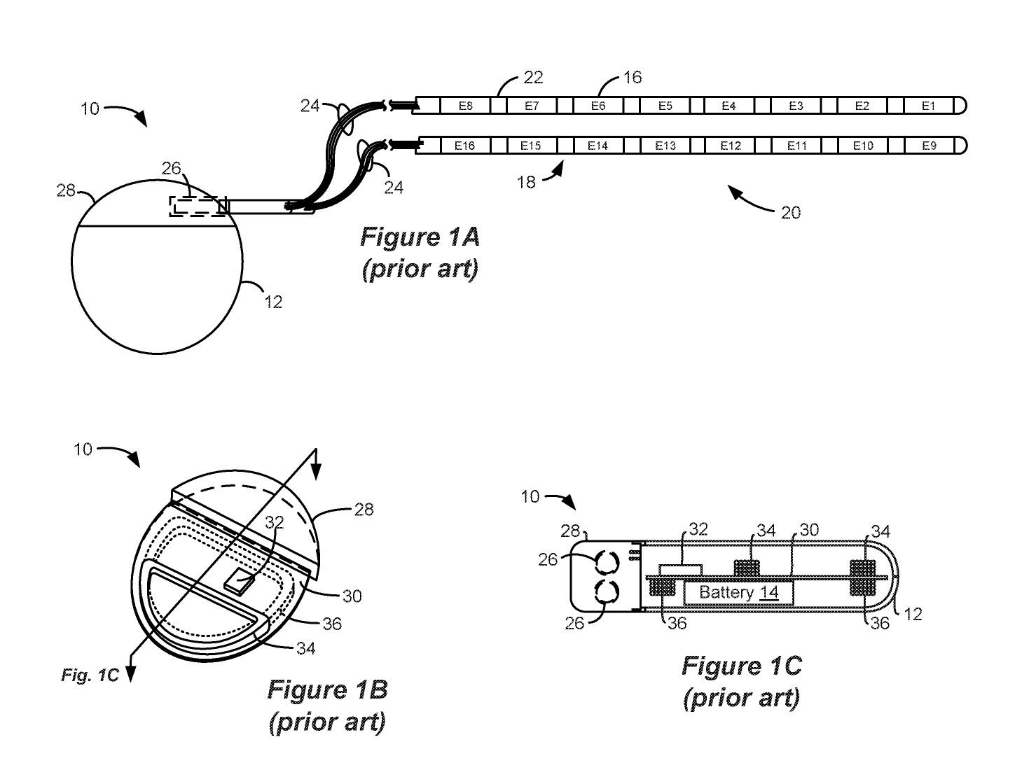

### DETAILED DESCRIPTION

The inventor has discovered that using one or more supercapacitors as a power source in an external charger and/or an implantable medical device (IMD) can significantly reduce the amount of time it takes a user to charge their IMD. The method can reduce the charging time from several hours down to one-two minutes or less.

Supercapacitors have a much greater power density than batteries, meaning that they can deliver higher power than a battery of comparable size/weight. That is because supercapacitors, such as hybrid supercapacitors, can be discharged at significantly higher rates than an equivalent sized Li-ion battery.

Discharge rates are often expressed as a C-rate. A C-rate is a measure of the rate at which a battery is discharged relative to its maximum capacity. A C-rate of 1 means that a battery can discharge the entire battery in one hour. A C-rate of C/2 means that a battery is capable of discharging half of the entire battery capacity in one hour. Li-ion batteries typically have a C-rate of about C/2 to C/4. In contrast, hybrid supercapacitors may have C-rates of about 30 C to about 60 C, meaning that they can discharge their entire capacity in one to two minutes. Additionally, hybrid supercapacitors can be charged very quickly, achieving 80% charge in under five minutes. Another advantage of hybrid supercapacitors over Li-ion batteries is that hybrid supercapacitors can be cycled many more times, typically thousands of cycles vs. hundreds of cycles for Li-ion batteries. Thus, using hybrid supercapacitors in the place of Li-ion batteries greatly extends the longevity of the device.

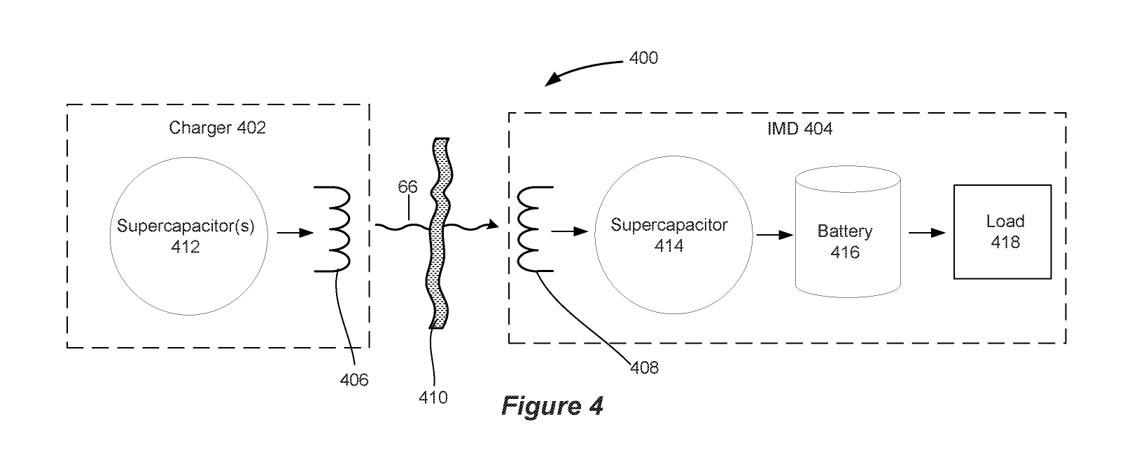

FIG. 4 illustrates a system 400 incorporating hybrid supercapacitors as power sources for both an external charger 402 and an IMD 404. As with the systems described above, the external charger 402 includes a charging coil 406 that inductively couples with a charging coil 408 of the IMD 404 through the patient's tissue 410. Rather than batteries, the external charger 402 includes one or more supercapacitors 412 that provide power to the coil 406. Since the supercapacitor(s) 412 have much higher discharge rates, as mentioned above, the supercapacitor(s) 412 can provide higher power to the coil 406 than would be possible with a comparable battery-powered external charger.

IMD 404 also includes a supercapacitor 414 configured to store power that is inductively transferred to the coil 408 from the coil 406 of the external charger 402. In the system 400, the power stored within the supercapacitor 414 can be used to charge a battery 416 within the IMD 404. The battery 416 is then used to power the “load” 418 of the IMD, i.e., the battery powers the circuitry required to operate the IMD 404. Alternatively, the power stored within the supercapacitor 414 can be used to power the load 418 directly, without the use of an intervening battery 416.

Since the coil 406 of the external charger 402 is powered using a supercapacitor 412 which has a much higher discharge rate C than a battery, the coil 406 can supply significantly more power over a shorter time to the coil 408 of the IMD 404 because of the greater discharge rate of the supercapacitor 412. And since the power received by the coil 408 of the IMD 404 is used to charge a supercapacitor 414 rather than a battery with the IMD 404, that charging time is also much shorter, due to the higher charging rate of the supercapacitor 414. Once the supercapacitor 414 is charged, the charging is completed, from the patient's perspective. Within the IMD 404, the power contained within the supercapacitor 414 can be used to charge the battery 416 “behind the scenes” at the battery's slower charging rate. Thus, the patient's experience with that the charging process takes only a few minutes, rather than nearly an hour.

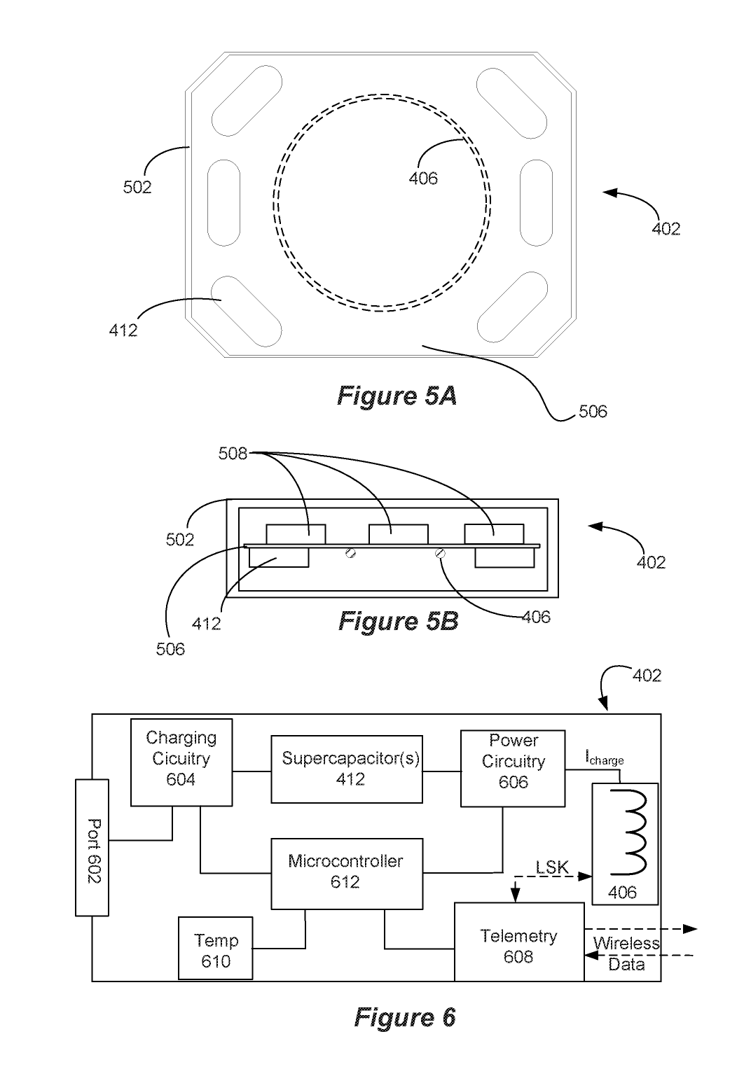

FIGS. 5A and 5B illustrate a cross-section views of an external charger 402 from the bottom and from the side, respectively. The external charger includes a housing 502, which may be of a hand-holdable form-factor. Alternatively, the external charger 402 may be comprised within a different form-factor, for example, a flexible belt that can be worn or blanket upon which a patient can sit or lay. Still alternatively, the external charger 402 may comprise multiple separate components. For example, the electronics and supercapacitor(s) 412 may be contained within a base unit, which may connect to a separate unit, such as a wand, which contains the charging coil 406. Various external charger configurations are known in the art.

The external charger 402 includes one or more supercapacitors 412. The embodiment of the external charger 402 illustrated in FIGS. 5A and 5B include six supercapacitors 412. Generally, any type of supercapacitor can be used, within design/size limitations. Examples of particularly suitable supercapacitors include lithium-ion or nickel-metal hydride hybrid supercapacitors. The illustrated external charger 402 includes six 1.4 V/90 F hybrid supercapacitors 412. The supercapacitors 412 may be wired in series to provide a higher total voltage or they may be wired in parallel. Such a configuration can provide about 20 watts of power for up to about two minutes. The supercapacitors may be mounted upon a printed circuit board (PCB) 506.

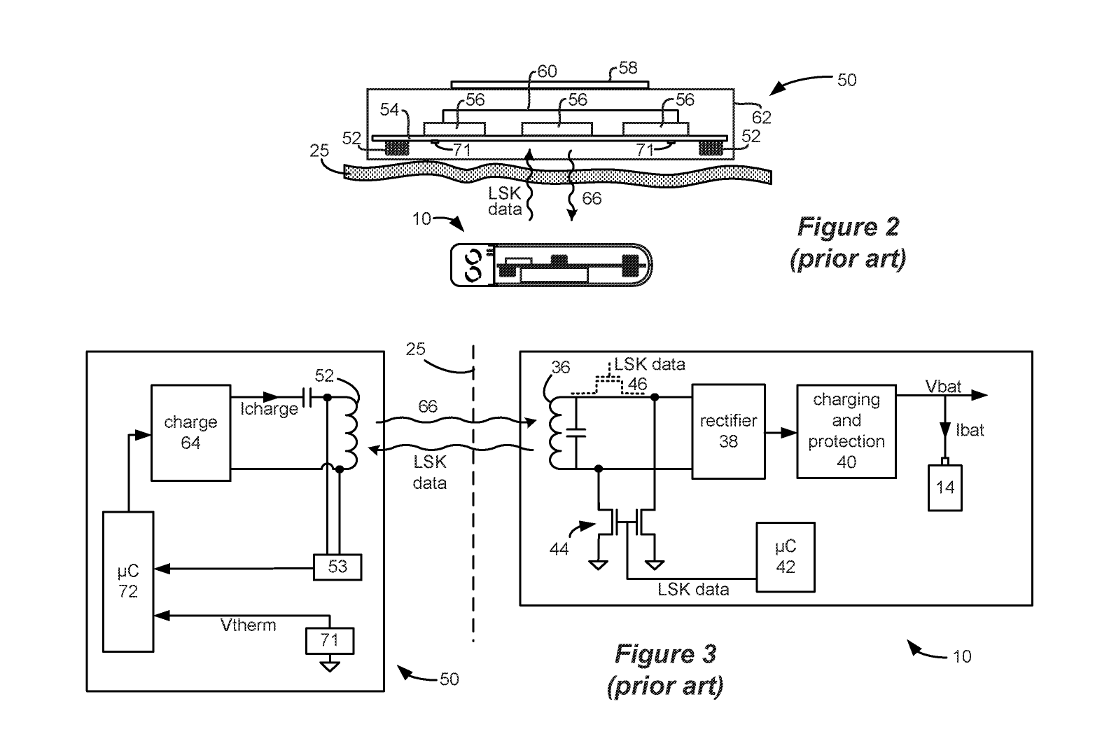

The external charger 402 also includes a charging coil 406 for inductively coupling with and transferring power to a coil in an IMD. Since the supercapacitors 412 provide a significant amount of current to the charging coil 406 over a short duration, it is important that to minimize the resistance of the charging coil 406 to increase the transmitted power. Charging coils used in battery powered external chargers, such as the prior art external charger 50 (FIGS. 2 and 3) typically comprise about 88 turns of 24 ga. Litz wire. In contrast, charging coil 406 may comprise fewer turns. The exact number of turns depends on the coil frequency, target power level, and coil size/construction. The coil conductor may be thicker, for example, 10 ga. to about 16 ga. Litz wire. The coil 406 may comprise 10-30 turns for frequencies up to approximately 1 MHz, for example. Fewer turns or solid copper tubing or rod may be used for higher frequencies, as discussed below. Alternatively, the charging coil 406 may be configured as a conductor trace upon the PCB 506. The external charger 402 also includes electronics elements 508 for controlling the operation of the external charger 402. Some of the electronics elements 508 are discussed in more detail below.

FIG. 6 shows a functional schematic of the external charger 402. The external charger 402 is typically configured with a port 602 (e.g., a USB port) to receive electric power for charging the supercapacitors 412. The port 602 may also allow data to be read from or programmed into the external charger 402, such as new operating software. The external charger 402 includes charging circuitry 604 for providing proper current and voltage for charging the supercapacitors 412. Excessive current or voltage can reduce the lifetime of supercapacitors. When charged with a constant current, a supercapacitor will hold a voltage that rises linearly with time. Supercapacitors can typically accept a wide range of charging currents, reducing the need for precision current control, but still requiring that charging stop when the device reaches its maximum rated voltage. Typically, charging is performed during an initial constant-current charging phase followed by a constant-voltage phase. During the constant-current phase, charging circuitry 604 may monitor output current by monitoring voltage across a sense resistor. An internal voltage regulator may provide precise control over the charging voltage. The charging circuitry 604 may monitor charging to each of the individual supercapacitors 412 and adjusts charging current/voltages to the supercapacitors to account for any imbalances.

The external charger 402 includes power circuitry 606 whereby energy stored in the supercapacitors 412 is used to energize charging coil 406 with AC current, I_{charge}. The power circuitry is discussed in more detail below. The external charger 402 further includes a telemetry module 608, which can receive and transmit telemetry data from and to an IMD. The telemetry data may include data relating to the temperature of the IMD, the amount of charge of the IMD's power supply (i.e., the supercapacitor and/or battery of the IMD), as well as other information. According to some embodiments, the telemetry module 608 may be configured to send/receive LSK data, as described in the introduction section above. According to some embodiment, the telemetry module 608 may be configured to send/receive wireless data, for example BlueTooth, WiFi, MICS, ZigBee, or another wireless protocol data.

The external charger 402 may further include a temperature sensor 610 configured to detect the temperature of the external charger 402 during charging. The temperature sensor 610 is a safety feature, allowing charging to be adjusted or interrupted if the temperature of the external charger 402 exceeds a level that is safe for the patient. The external charger 402 further includes a microcontroller 612 that controls aspects of the operation of the external charger 402, as explained in more detail below. It should be noted that the microcontroller 402 may further include one or more user interface (UI) modalities (not shown), such as buttons, LED lights, speakers, and/or a graphical user interface, whereby the patient interacts with and controls the external charger 402. The external charger 402 may include other features known in the art, such as alignment indicators, for example.

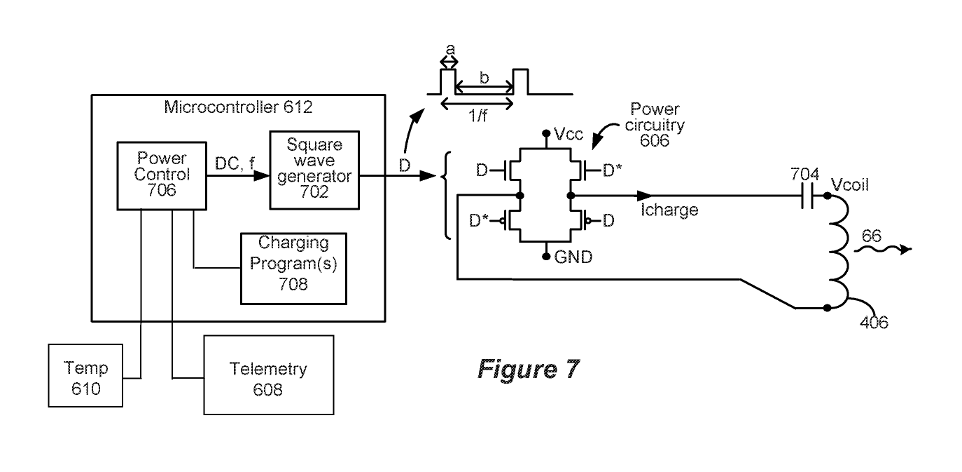

FIG. 7 shows further details regarding the power circuitry 606 used to energize the charging coil 406 with AC current, Icharge. A digital drive signal D is formed by a square wave generator 702, which may comprise a part of the control circuitry within the microcontroller 402 or may act under the direction of the microcontroller 402. Drive signal D comprises a pulse-width modulated (PWM) signal with a periodically-repeating portion that is high (logic ‘1’) for a time portion ‘a’ and low for a time portion ‘b’. As such, the drive signal D has a duty cycle DC equal to a/(a+b). Further, the drive signal D has a frequency f equal to 1/(a+b). The frequency f of the drive signal is generally set to or near the resonant frequency of the capacitor 704/charging coil 406 LC circuit.

The AC voltage V_{coil} induced across the charging coil 406 will oscillate at a frequency of f, as determined by the power circuitry 606. In battery-operated systems, such as described in the introduction above, the oscillation frequency f is typically around 80 kHz. In the supercapacitor operated external charger 402, the oscillation frequency f may be higher. Higher frequencies can be more efficient for power transfer. For example, the charging oscillation frequency f may be greater than 1 MHz. For example, the oscillation frequency may be in the range of 6-7 MHz or in the range of 13-14 MHz. According to some embodiments, the charging oscillation frequency is 6.78 MHz, which is the power transmission band corresponding to the Alliance for Wireless Power (A4WP) standard. According some embodiments, the charging oscillation frequency is 13.56 MHz, which is reserved for industrial, scientific and medical (ISM) purposes. The frequency of the drive signal can also be adjusted, as explained subsequently, and may include frequencies outside of those bands.

Power circuitry 606 can comprise a well-known H-bridge configuration, including two P-channel transistors coupled to a power supply voltage Vcc, and two N-channel transistors coupled to a reference potential such as ground (GND). According to some embodiments, the transistors may be silicon-based metal-oxide-semiconductor field-effect transistors (MOSFETs). According to some embodiments, the transistors may be Gallium nitride (GaN) field-effect transistors (GaNFETs), which can operate much faster and have higher switching speeds than traditional MOSFETs. The transistors are driven on and off by the drive signal D and its logical complement D*. In so doing, the power supply voltage Vcc and ground are made to alternate across the LC circuit t frequency f, thus producing the magnetic charging field 66 at this frequency. Power supply voltage Vcc may comprise the voltage of the supercapacitors 412 (FIG. 6) in the external charger 402, or may be regulated from that voltage. As is well known, the duty cycle DC of the drive signal D can be increased from 0 to 50% to increase Icharge, thus setting the power at which the charging coil 406 is energized and hence the power of the resulting magnetic field 66.

The power transmitted by the magnetic field can be controlled by power control circuitry 706. Power control circuitry 706 can operate as firmware within the microcontroller 402, although this is not strictly necessary as analog circuitry can be used for certain aspects as well. The power control circuitry 706 determines the amount of DC power and/or the frequency f provided to the power circuitry 606, generally, with the goal of maximizing the power of the charging magnetic field 66 (thereby minimizing charging time) within the limits of comfort and safety to the patient.

Since significantly more power is transmitted via the magnetic field 66 in the supercapacitor-operated external charger 402 than in the battery-operated systems described in the introduction section above, temperature control can be crucial. Thus, the power control circuitry can receive, as input data, data from the temperature sensor 610 of the external charger 402 as well as data relating to the temperature measured in the IMD and transmitted to the telemetry module 608 of the external charger 402. It should be noted, that although the higher energy transfer rates obtainable using the supercapacitor-based charging system generate higher temperatures, the potential deleterious impact of those higher temperatures are somewhat offset by the significantly shorter charging times. The relevant technical standards for the safety and effectiveness of medical electrical equipment published by the International Electrotechnical Commission (IEC 60601-1) allow a temperature of up to 60° C. for up to one minute, up to 48° C. if between one and ten minutes, and up to up to 43° C. for greater than ten minutes.

The power control circuitry 706 can be programmed to adjust the power to the coil to maintain temperatures of the IMD and of the external charger 402 within those guidelines, generally, by adjusting the duty cycle of the charging. If data received from either the temperature sensor 610 or the telemetry module 608 indicates a temperature that is too high, then the power control circuitry 706 can interrupt charging or decrease the charging rate, typically by decreasing the duty cycle. The power control circuitry 706 can also be controlled by one or more charging programs 708 configured to maximize the charging rate within safety parameters. The charging programs 708 may be operable as software or firmware. One such charging program 708 may instruct the power control circuitry 706 to control charging as a pulsed charging sequence, whereby the charging coil 406 is powered for several seconds and is then idle for several seconds. Another charging program 708 may be a ramped charging program, whereby the duty cycle is initiate at a high value and ramped down to a lower value as a function of time. Still alternatively, the duty cycle may alternate between high and low values, to maximize charging while maintaining safe temperatures within the external charger 402 and within the IMD. Once the IMD's power source is charged to capacity, the IMD may send data to the telemetry module 608 indicating such, whereupon the power control circuitry 706 may end the charging. According to some embodiments, the microcontroller 612 may cause the external charger 402 to inform the patient that charging is completed.

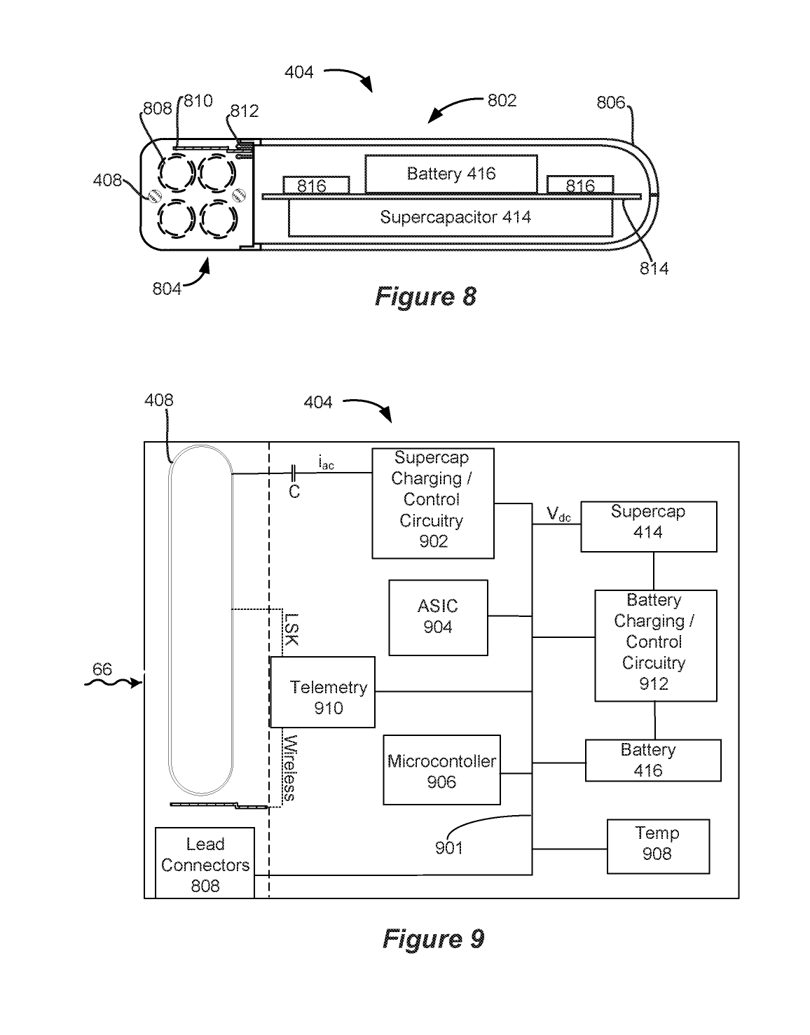

FIG. 8 illustrates a supercapacitor-powered IMD 404. The IMD includes a case 802 and a header 804. The case 802 contains the electronics for powering and operating the IMD 404 and typically comprises a housing 806 formed of a biocompatible metallic material such as titanium. The header 804 typically comprises a non-metallic material, such as epoxy, for example. The header 804 contains one or more lead connectors 808 for attaching to leads, such as leads 18 described in the Introduction section above. The illustrated IMD 404 includes four lead connectors 808. The header 804 may also contain a wireless antenna 810 for transmitting wireless data between the IMD 404 and an external charger 402. Electric communication between the header 804 and components within the case 802 is provided by electric feedthroughs 812. The header 804 of the illustrated IMD 404 also contains a charging coil 408. While the charging coil may be contained within the case, according to some embodiments may be preferable that the charging coil 408 is configured within the header 804, because of the high charging frequencies that may be used. For example, embodiments of the IMD 404 are charged using a magnetic field 66 having frequencies greater than 1 MHz, which cannot efficiently penetrate the metallic case 806.

The case 802 of the IMD 404 includes a PCB 814, upon which may be supported a battery 416, circuitry 816, and a supercapacitor 414. The supercapacitor 414 may generally, any type of supercapacitor, within design/size limitations. Examples of particularly suitable supercapacitors include lithium-ion or nickel-metal hydride hybrid supercapacitors. The illustrated IMD 404 includes a 1.4 V/90 F hybrid supercapacitor. The battery 416 is typically a rechargeable battery, such as a 4.2 V Li-ion battery.

FIG. 9 shows a functional schematic of an IMD 404. Components of the IMD 404 may communicate with one another via one or more busses 901. The IMD 404 includes supercapacitor charging circuitry 902, which charges the supercapacitor 414 using the AC current i_{ac} induced in the coil 408 by the magnetic field 66 received from the external charger. The AC current i_{ac} may be filtered by a capacitor C. The supercapacitor charging circuitry 902 rectifies received current and may include a voltage-magnitude-limiting Zener diode (as known in the art) to establish a DC voltage, V_{dc} for charging the supercapacitor 414. Portions of the supercapacitor charging/control circuitry 902 may reside on an Application Specific Integrated Circuit (ASIC) 904. The ACIC 904 may comprise additional circuitry necessary for operating the IMD 404, such as generating current to the various electrodes connected to the lead connectors 808, determining telemetry, controlling system memory, etc. Portions of the supercapacitor charging/control circuitry 902 may also comprise off-chip components, such as capacitor C and other active or passive components.

The supercapacitor charging/control circuitry 902 may be configured to determine the charge state the supercapacitor 414, generate the appropriate current for charging the supercapacitor 414, initiate charging, and terminate charging when the supercapacitor reaches an appropriate voltage. Such functions may be controlled by a microcontroller 906. Examples of suitable microcontrollers include Part Number MSP430, manufactured by Texas Instruments, which is known in the art.

The IMD 404 may monitor the temperature of the IMD 404 during charging using a temperature sensor 908. Should the temperature of the IMD 404 approach or exceed certain temperature limits, the IMD 404 (via the microcontroller 906) may instruct the external charger 402 (FIG. 6) to cease or adjust the amount of magnetic charging field 66, as described above. The IMD 404 may communicate with the external charger 402 via a telemetry module 910. As described above with respect to the external charger 402, the telemetry module 910 of the IMD 404 may be configured to send/receive LSK data, as described in the introduction section above. According to some embodiment, the telemetry module 910 may be configured to send/receive wireless data, for example BlueTooth, WiFi, MICS, ZigBee, or another wireless protocol data.

Further regarding temperature management within the IMD 404, some embodiments of the IMD 404 include heat-sinking architectures. For example, some embodiments of the IMD 404 include added thermal mass comprising a metal, such as copper, for heat-sinking. Some embodiments may comprise phase change materials, such as known in the art for heat management.

The supercapacitor charging/control circuitry 902 monitors the charging of the supercapacitor 414 to determine when the supercapacitor is fully (or adequately) charged. Once the supercapacitor 414 is charged, the IMD 404 may transmit a signal to the external charger 402 via the telemetry module 910 informing the patient that charging is complete. As mentioned above, charging the supercapacitor 414 may take only a matter of minutes. Once the supercapacitor is charged, battery charging/control circuitry 912 can be implemented (for example, controlled by a microcontroller 906) to cause the charge stored in the supercapacitor 414 to charge the battery 416. The charging of the battery 416 may occur “off line,” in the sense that it is not apparent to the patient. In other words, from the patient's perspective, charging is completed once the supercapacitor 414 is charged.

The battery charging/control circuitry 912 may be implemented as circuitry on the ASIC 904 and/or as off-chip circuitry. The battery charging/control circuitry 912 may perform several functions. For example, the battery charging/control circuitry 912 may step up the voltage from the supercapacitor 414 to a voltage adequate to charge the battery 416. The embodiment illustrated in FIGS. 8 and 9 may include a 1.4 V/90 F supercapacitor 414 and a Li-ion battery 416 of about 4.2 V, for example, as mentioned above. In such a case, the battery charging/control circuitry 912 may include a voltage boost stage, which boosts the voltage available from 1.4 V to a voltage greater than 4.2 V. The voltage boost circuitry may comprise a capacitor-based charge pump, an inductor-based boost converter, or any other DC-DC voltage converter known in the art. Ultimately, the battery charging/control circuitry 912 regulates the charging and control of the battery 416. The battery charging/control circuitry 912 may detect when the battery 416 needs charging and may cause the IMD 404 to telemeter that information to the external charger via the telemetry module 910. The battery 416 is used to power the IMD 404, including powering therapy and monitoring functions, as is known in the art.

It will be apparent to a person of skill in the art that the “hybrid power” system of the IMD 404 affords the patient a significantly improved user experience. The supercapacitor 414 can be charged at a high rate, as high as 30 C in some cases. For example, the supercapacitor charging rate may be 20 C to 30 C. The energy stored in the supercapacitor 414 is then slowly discharged to the battery 416 at the slower battery charge rate, for example C/4 to C/2. The slower battery charging rate, which occurs in the background, can significantly increase the battery's longevity. Since the energy density of the supercapacitor 414 may be less than that of the Li-ion battery 416, multiple charging sessions may be needed to fully charge the battery. However, each of those sessions require less time than simply charging the battery directly. If the battery 416 is fully charged, the supercapacitor can provide additional energy capacity beyond that of the Li-ion battery. According to other embodiments, the IMD 404 may not include a battery 416 and related circuitry, in which case the IMD 404 functionality is powered using the supercapacitor 414.

It will be appreciated that the IMD 404 is particularly suited to be charged using the supercapacitor-powered external charger 402 since the supercapacitors of the external charger 402 are configured to supply a high magnetic field for rapid charging. However, generally any type of external charger capable of producing a high magnetic field may be used to charge the IMD 404. For example, an external charger may be powered using a wall outlet or a standalone power supply, as is known in the art.

Although particular embodiments of the present invention have been shown and described, it should be understood that the above discussion is not intended to limit the present invention to these embodiments. Referring to “a” structure in the attached claims should be construed as covering one or more of the structure, not just a single structure. It will be obvious to those skilled in the art that various changes and modifications may be made without departing from the spirit and scope of the present invention. Thus, the present invention is intended to cover equivalents that may fall within the spirit and scope of the present invention as defined by the claims.

What is claimed is:

1. An implantable medical device (IMD), comprising:
a supercapacitor;
a coil configured to receive a magnetic field;
a first circuitry configured to convert current induced in the coil by the magnetic field into a voltage and to charge the supercapacitor with the voltage; and
a second circuitry configured to charge the rechargeable battery from charge stored within the supercapacitor.

2. The IMD of claim 1, wherein the supercapacitor is a hybrid supercapacitor.

3. The IMD of claim 1, wherein the magnetic field has an oscillation frequency of greater than 1 MHz.

4. The IMD of claim 3, wherein the oscillation frequency is in the range of 6-7 MHz or in the range of 13-14 MHz.

5. The IMD of claim 1, further comprising a rechargeable battery.

6. The IMD of claim 1, wherein the first circuitry charges the supercapacitor at a rate of about 20 C to about 30 C.

7. The IMD of claim 1, wherein the second circuitry charges the rechargeable battery of at a rate of about C/4 to about C/2.

8. The IMD of claim 1, wherein the rechargeable battery provides power for stimulation therapy.

9. An external charger for an implantable medical device (IMD), the external charger comprising:
a coil,
one or more supercapacitors, and
a circuitry configured to power the coil using power stored within the one or more supercapacitors, causing the coil to generate a magnetic field.

10. The external charger of claim 9, wherein the coil comprises a metal trace upon a printed circuit board (PCB).

11. The external charger of claim 9, wherein the one or more supercapacitors comprise hybrid supercapacitors.

12. The external charger of claim 9, wherein the magnetic field has an oscillation frequency of greater than about 1 MHz.

13. The external charger of claim 9, wherein the circuitry is configured to cause the coil to generate a magnetic field having a first power and to cause the coil to generate a magnetic field having a second power, which is less than the first power.

14. The external charger of claim 13, wherein the circuitry is configured to switch from the first power to the second power in response to data received from the IMD.

15. The external charger of claim 14, wherein the data received from the IMD comprises data relating to temperature of the IMD.

16. A system for providing therapy to a patient, the system comprising:
an implantable medical device (IMD) comprising:
a first supercapacitor,
a rechargeable battery,
a first coil configured to receive a magnetic field,
a first circuitry configured to convert current induced in the first coil by the magnetic field into a voltage and to charge the supercapacitor with the voltage, and
a second circuitry configured to charge the rechargeable battery from charge stored within the supercapacitor, and
an external charger for providing a magnetic field to charge the IMD.

17. The system of claim 16, wherein the external charger comprises:
a second coil,
one or more second supercapacitors, and
a third circuitry configured to power the second coil using power stored within the one or more second supercapacitors, causing the second coil to generate the magnetic field.

18. The system of claim 17, wherein the magnetic field has an oscillation frequency of greater than about 1 MHz.

19. The system of claim 17, wherein the one or more second supercapacitors discharge at a rate of about 20 C to about 30 C.
